(19) United States
(12) Patent Application Publication
(10) Pub. No.: US 2016/0081838 A1
(43) Pub. Date: Mar. 24, 2016

(54) ANKLE SUPPORT BRACE (ASB) AND METHOD OF MAKING THE SAME

(71) Applicants: Frank J. Ledezma, Brookfield, WI (US); Bracemasters International, LLC, New Berlin, WI (US)

(72) Inventors: Frank J. Ledezma, Brookfield, WI (US); Timothy F. O'Keefe, Delevan, WI (US)

(21) Appl. No.: 14/860,268

(22) Filed: Sep. 21, 2015

Related U.S. Application Data
(60) Provisional application No. 62/053,591, filed on Sep. 22, 2014.

(51) Int. Cl. A61F 5/01

(52) U.S. Cl. CPC A61F 5/0111; A61F 5/0127

(57) ABSTRACT

The present disclosure provides a custom or pre-fabricated ankle support brace providing tri-planar control of the ankle foot structure and allows for free plantar dorsi flexion. The ankle support brace comprises a stirrup-shaped brace body composed of reinforcement strips and a closure mechanism.

### FIELD

This disclosure relates to providing a custom or pre-fabricated ankle support brace (ASB). The ASB provides medial-lateral support yielding varus and valgus control to protect from excessive rotation or extreme medial-lateral motion that could go beyond normal end-range of motion, causing injury. The ASB protects against ankle instability or injury during normal or extreme human locomotion while allowing free plantar and dorsi flexion as well as tri-plane motion.

### BACKGROUND

Typical ankle braces use flexible materials to provide a comfortable fit and little, if any, motion restriction, and thus are very limited in support. These ASBs may not prevent injury in excessive activity associated with physical exercise programs or athletic events. ASBs which do include a combination of fabric cover with rigid or semi-rigid inserts, provide support but severely limit ankle mobility. In addition, current ASBs suffer from lack of durability, especially if the person using them is involved in aggressive athletic activity.

In response to the limitations of such ASBs, the athletic industry typically uses adhesive tape to protect athletes since it provides an adequate support, but often limits mobility. However, adhesive tape is not durable and often breaks down during use. The tape is generally not reusable. Still further, before every athletic event a person needs to take the time to tape their ankle.

There exists a need in improving conventional ASBs to treat acute or chronic ankle conditions without either limiting mobility or failing to provide sufficient support, while offering a level of comfort and durability suitable for intense, athletic applications that can be worn inside common civilian or athletic footwear. The improved ankle brace would be durable, reusable and provide the correct balance between support and mobility when used in conjunction with a shoe.

### SUMMARY

The present disclosure provides an ankle support brace which has a stirrup-like shape comprising a combination of a closed cell polyethylene foam inner lining bonded to a polyolefin elastomeric material, with one or more key structural thermoplastic components situated between the polyethylene foam inner lining and the outer layer of polyolefin elastomeric material. In an embodiment, a key structural thermoplastic component is wrapped around the posterior half of the proximal edge of the ankle support brace, and a second key structural thermoplastic component is structured as a stirrup-like configuration that extends as a unitary element continuously from the upper medial side to under the mid-foot and back up to the upper lateral side of the brace.

Not only does the present disclosure provide an ASB composed of a combination of specific materials varied in stiffness and texture, but it provides a specific relative amount and placement of key structural thermoplastic components to yield an ankle support brace having an improved combination of support, flexibility and comfort that can be tailored to better meet the specific needs of each individual patient.

In one embodiment, the disclosure provides an ASB arranged to provide varus and valgus control and medial-lateral inversion or eversion control comprising a brace body comprising a medial side portion, a lateral side portion, a medial front edge of the medial side portion, a lateral front edge of the lateral side portion, a sole portion in a plane at least partially horizontal to the medial and lateral side portions having an arch portion and a heel portion, and a back portion extending from the heel portion to a lower calf portion. In embodiments, the brace body comprises a minimum of three layers including a closed cell polyethylene foam inner layer, a rigid or semi-rigid thermoplastic intermediate layer as key structural thermoplastic components, and a polyolefin elastomeric material as an outer layer. Optionally, an additional material layer can be applied over the surface of the foam inner layer to enhance the texture and provide a soft interface with the foot, for example, a synthetic leather-like material or elastic fabric.

In an embodiment, the ASB is stirrup shaped and comprises a medial stirrup-type reinforcement strip with a first end situated a distance below the proximal edge of the orthosis and extending down along the medial side portion to under the mid-foot, and continuing up the lateral side with a second end situated below the proximal edge of the brace. The brace further comprises a proximal reinforcement strip beginning on the medial side just below the proximal edge and extending around the posterior side to an end point on the lateral side. The brace further includes a closure mechanism positioned on the lateral side end of the proximal reinforcement strip and a closure mechanism positioned on the medial side end of the reinforcement strip. Multiple closure mechanisms can be situated on the lateral anterior edge and the medial anterior edge of the stirrup type reinforcement strip wherein the closure mechanisms are structured and operable to tighten and secure the ASB onto the subject's lower leg and foot.

The present disclosure also provides a method of manufacturing an ASB. In an embodiment, the method comprises the steps of providing a mold of a lower extremity, forming a closed cell polyethylene foam material over the mold, cutting away portions of the foam to create openings at the heel and at the foot portion forward of the metatarsals, forming at least one key structural thermoplastic component over the closed cell polyethylene foam to bond to the foam as structural reinforcements, forming an outer layer of a polyolefin elastomeric material over the prior two layers on the mold to create a third bonded layer, setting closure mechanisms (e.g., lace loops) into the polyolefin elastomeric outer layer, and removing the mold from the material layers to produce the ASB.

The ankle support brace of the disclosure is an improved alternative to traditional ankle braces, providing a broad range of features and benefits that address the limitations of traditional braces and leather ankle gauntlets. The present thermoplastic ankle support brace is hygienic, waterproof, easily cleaned, and is readily adjustable at fitting utilizing a modest heat application. The present device provides more flexible comfort with complete control, and is more durable than traditional ASBs. The device has a slim, low profile design that fits inside most shoes. The improved ASB provides a soft interface with the patient's foot to enhance comfort and alternatives for closure mechanisms.

### BRIEF DESCRIPTION OF THE DRAWINGS

Preferred embodiments of the disclosure are described below with reference to the following accompanying drawings, which are for illustrative purposes only. Throughout the following views, the reference numerals will be used in the drawings, and the same reference numerals will be used throughout the several views and in the description to indicate the same or like parts.

FIG. 1 is a perspective view of an embodiment of an ankle support brace according to the disclosure.

FIG. 2 is a perspective view of another embodiment of an ankle support brace according to the disclosure.

FIG. 3 is a medial side view of the ankle support brace of FIG. 1 positioned inside a shoe (shown in phantom).

FIG. 4 is a cross-sectional view of the ankle support brace of FIG. 1, taken along line 4-4 of FIG. 1.

FIG. 5 is an exploded view of the ankle support brace of FIG. 1, showing the inner layer situated on a mold (shown in phantom) and two structural components.

FIG. 6 is a perspective view of the ankle support brace of FIG. 5, in a subsequent process step showing the key structural components on the inner layer and the applied outer layer.

FIG. 7 is a block diagram of an embodiment of a process for making an ankle support brace according to the disclosure.

### DETAILED DESCRIPTION

Embodiments of the disclosure relate to an ankle support brace (ASB) and methods of making the orthosis. The ASB is arranged to provide varus and valgus control and stabilize the talus (subtalar and mid-tarsal joints). The ASB of the disclosure helps protect against ankle weakness and/or instability of the ankle-foot structure.

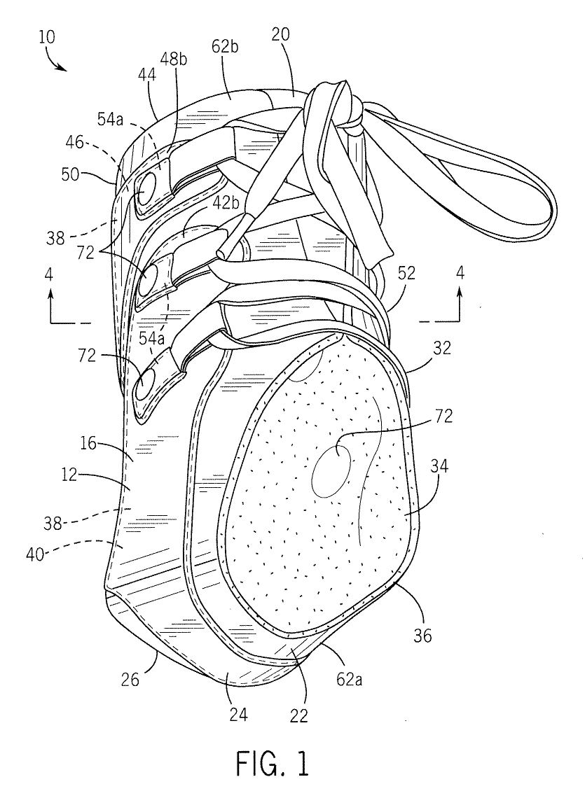

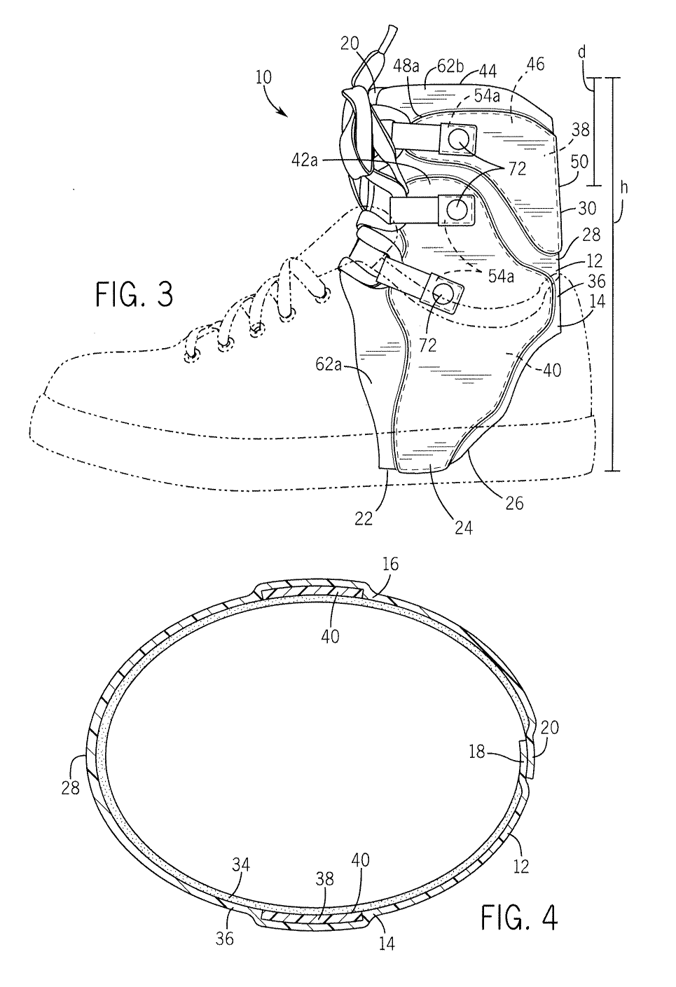

An embodiment of an ankle support brace 10 according to the disclosure is described with reference to FIGS. 1, 3 and 4. The brace can be used in combination with a standard shoe (shown in phantom in FIG. 3). The brace is designed to be worn over a sock and inside the shoe without the need to increase the shoe size to accommodate the brace. The brace is in the general shape of a stirrup design without the heel portion of the boot and without the portion that would be forward of the mid-tarsal joint.

Referring to FIGS. 1, 3 and 4, the ankle support brace 10 comprises a stirrup-shaped brace body 12 having a medial side portion 14, a lateral side portion 16, a medial front edge 18 of the medial side portion 14, a lateral front edge 20 of the lateral side portion 16, a sole portion 22, an arch portion 24, a heel opening edge 26 (forming a heel opening), and a back portion 28 extending from the heel opening edge 26, forming a heel opening, to an upper calf portion 30. The sole portion 22 is in a plane at least partially horizontal to the medial and lateral side portions 14, 16 of the brace body 12. As can be seen, at least a portion of the sole portion 22 is at least partially perpendicular to the medial and lateral side portions 14, 16 of the brace body 12. The ankle support brace 10 further includes a closure mechanism 32 to tighten and secure the orthosis to the foot. FIGS. 1 and 3 are lateral and medial side views, respectively, of the ankle support brace 10.

As illustrated, the brace body 12 comprises a laminated structure of at least three material layers including an inner layer 34, a polyolefin elastomeric outer layer 36, and the rigid or semi-rigid key structural components as an intermediate layer 38 between the inner and outer layers 32, 34.

The inner layer 34 is composed of a closed cell polyethylene (PE) foam, which, in embodiments, is at least 1.5 mm thick, preferably at least 3.0 mm thick, up to 4.5 mm thick. Closed cell PE foams are commercially available, for example, under the tradenames Volara® from Sekisui Voltek, LLC (Lawrence, Mass.), and Aliplast from AliMed, Inc. (Dedham, Mass.). Sheets of closed cell polyethylene foam are commercially available.

The outer layer 36 is composed of a rigid or semi-rigid thermoplastic material, preferably a polyolefin elastomeric material having thermoforming capabilities. In embodiments, the outer layer 36 is at least 2.0 mm thick, preferably at least 3.0 mm thick, up to 5.0 mm thick. In an embodiment, the polyolefin elastomeric material is an ethylene-butene copolymer or an ethylene-octene copolymer having a melt index range at 190° C. of less than 0.5 to 30 g/10 min (measured according to ASTM D 1238), a density of 0.857 to 0.910 g/cm³ (measured according to ASTM D 792), a melting range of 36° C. to 104° C., a Shore A Hardness of 56 to 96 (ASTM D 2240), and a flexural modulus from 3 to 110 MPa (measured according to ASTM D 790). In another embodiment, the polyolefin elastomeric material is an ethylene-butene copolymer having a density of 0.885 g/cm³ (measured according to ASTM D 792), a melt index of 1.0-2.5 g/10 min (2.16 kg @ 190° C. measured by ASTM D 1238), a Mooney Viscosity of 13 (ML 1+4 @121° C., measured according to ASTM 1646), a Shore A durometer hardness of 80-84 (measured according to ASTM 2240) and an ultimate tensile strength of 9.3-11.2 MPa (508 mm/min measured according to ASTM D 638). Polyolefin elastomers are well known and commercially available, for example, ENGAGE® ethylene/α-olefin copolymers available from The Dow Chemical Company.

The ankle support brace 10 further includes an intermediate layer 38 defined by the key structural components and composed of a thermoplastic, polyolefin elastomeric material that is preferably at least 1.5 mm thick, preferably at least 3.0 mm thick, up to 4.5 mm thick. The key structural thermoplastic components are laminated (or inset) into the orthosis to provide support in target areas without reducing the flexibility of the brace body 12 in other areas. The intermediate layer 38 for the key structural components is preferably stiffer than the thermoplastic material used for the outer layer 36 of the brace body 12. Nonlimiting examples of suitable thermoplastic polyolefin elastomers include polypropylene, polyethylene and modified polyethylene (MPE), which are commercially available from a number of sources.

As shown in FIG. 1, a first key structural component is configured as a medial stirrup-type reinforcement strip 40 in one continuous strip with a first end 42a positioned below the proximal edge 44 of the brace body 12 on the medial side portion 14, preferably at a distance (d) of about one-fifth (or 20%) of the total height (h) of the brace body from the proximal edge 44. The reinforcement strip 40 extends to under the sole portion 22 at the arch portion 24 (at about mid-foot), and continues up the lateral side portion 16 with the second end 42b positioned below the proximal edge 44 of the brace body 12, preferably at a distance (d) of about one-fifth (or 20%) of the total height (h) of the brace body 12 from the proximal edge 44.

In addition, a second key structural component is configured as a proximal reinforcement strip 46 positioned just below the proximal edge 44 of the brace body 12, with a first end 48a positioned on the medial side portion 14 and extending around the posterior side portion 50 with a second end 48b positioned on the lateral side portion 16 of the brace body 12.

The first key structural component (40) (stirrup reinforcement strip 40) stabilizes the talus and provides varus and valgus control. The second key structural component (proximal reinforcement strip 46) provides additional circumferential rigidity and an attachment point for closure mechanisms. When the ankle support brace 10 is fastened on the foot, the first and second key structural components work in conjunction with each other to provide varus and valgus control.

The closure mechanism 32 may include laces with a series of holes, eyelets, loops or hooks, Velcro® strips (available from Velcro USA, Inc.), elastic closures, cinched straps, zippers, snaps, buttons, hooks, clasps or other suitable fastener. In embodiments, when the closure mechanism 32 is engaged, the medial front edge 18 and the lateral front edge 20 of the brace body overlap, as shown in FIG. 4, such that the brace body does not require a separate tongue element. The overlap can be from 0.125 inches to 1.5 inches (0.32 cm to 3.8 cm), for example, 0.25 inch to 1.0 inch (0.64 cm to 2.5 cm).

As illustrated in FIG. 1, in an embodiment, the closure mechanism 32 is in the form of a lace 52 that cooperates with two or more lace loops 54a positioned on the stirrup reinforcement strip 40 at the proximal section 62b (which would typically reside outside the shoe) of the brace body 12 to engage the lace 52 and tighten and secure the ankle support brace 10 onto the subject's foot. Because distal section 62a (or the region that would typically reside inside the shoe) is cut off so as to be toe-less and footless forward of the metatarsals, the distal section 62a does not include any closure mechanisms. In further embodiments, metallic or non-metallic “boot hook” style hardware can be installed in the stirrup-type reinforcement strip 40 to engage with the lace 52.

As shown in FIG. 3, the stirrup-like shape of the ASB 10 results in reduced material in the shoe compared to conventional boot-style braces. The reduced material in the shoe improves wearer mobility, particularly during athletic activity. The first key structural component (40) (stirrup reinforcement strip 40) and second key structural component (proximal reinforcement strip 46 provide support, stabilize the talus and provide varus and valgus control, and the overall design of the ASB 10 provides an enhanced element of control around the Achilles. As a result, the ASB 10 provides a balance of support and protection without hindering mobility.

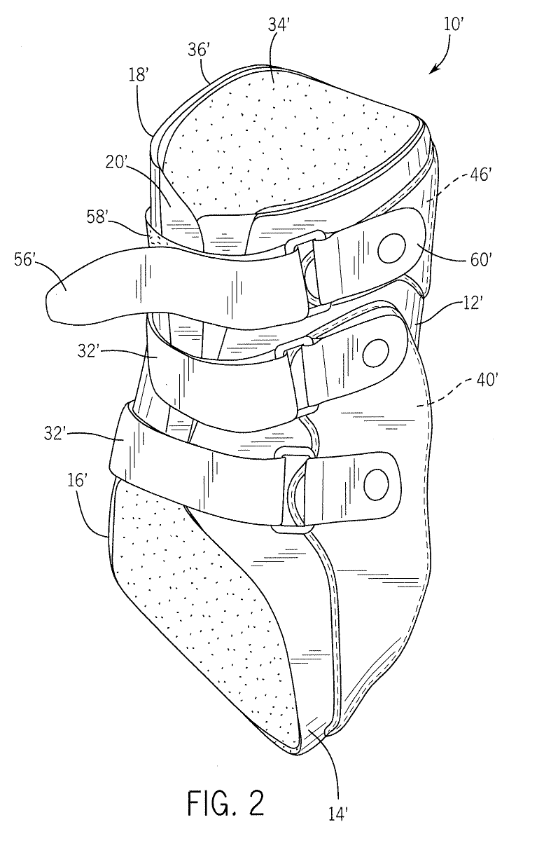

In another embodiment shown in FIG. 2, the closure mechanism 32′ is a hook and loop fastening mechanism in the form of an attachment strap 56′ as a first Velcro part with a one end secured to the proximal reinforcement strip 46′ and the stirrup reinforcement strip 40′ on the lateral side portion 16′ of the brace body 12′, and a loop element 60′ secured′ to proximal reinforcement strip 46′ and the stirrup reinforcement strip 40′ on the medial side portion 14′. The second end of the attachment strap 56′ is inserted through the loop of the loop element 60′, pulled back and attached to a cooperating second Velcro part 58′ to tighten the ankle support brace 10′ onto the person's foot and ankle.

In some embodiments, the brace body 12 further includes an interface layer (not shown) that is applied over the foam inner layer 34 to enhance and provide a soft interface with the subject's foot. The interface layer can be, for example, a synthetic leather-like material (e.g., Clarino® artificial leather), a synthetic elastic fabric (e.g., Lycra® material), or other soft material.

Optionally, a layer of a synthetic leather-like material (e.g., Clarino® artificial leather) or a synthetic elastic fabric (e.g., Lycra® material) can be applied over the surface of the foam inner layer, or a pre-laminated polyethylene foam with a bonded synthetic layer, to provide an interior interface with the foot to enhance the texture of the soft interface.

### Method of Manufacture

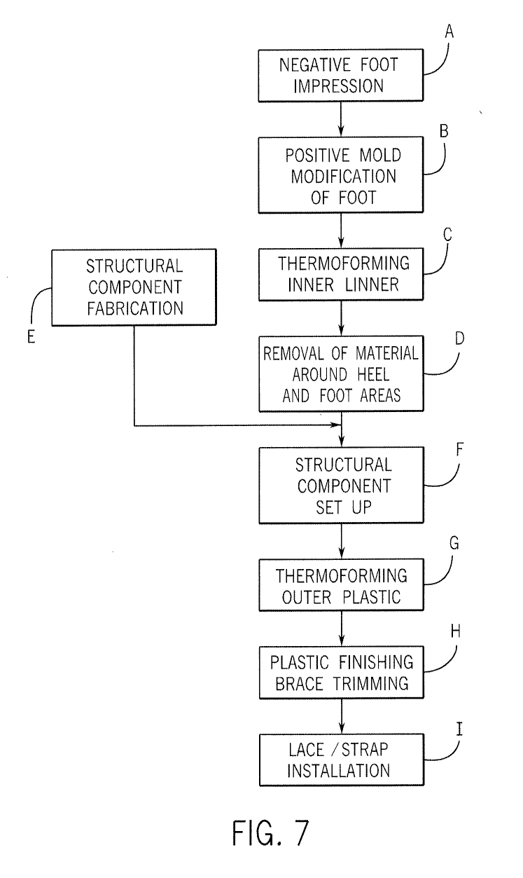

FIG. 7 provides a process flow diagram of a method of manufacture of an ankle support brace according to the disclosure.

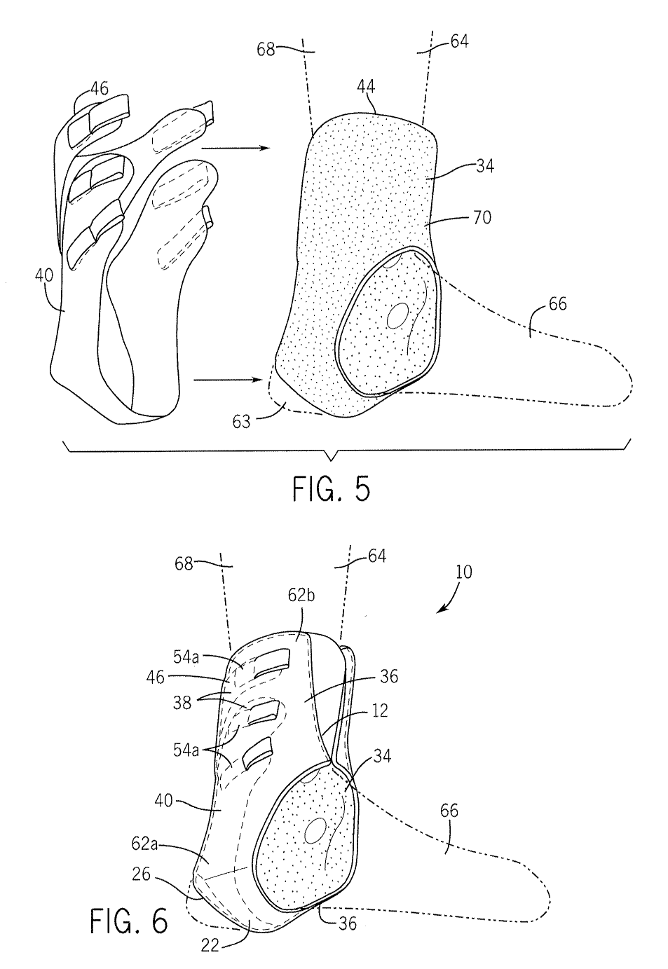

In first steps A and B, a mold 64 (e.g., plaster, wax, metal, wood, epoxy molds) of a patient's lower extremity is provided, as shown in phantom in FIG. 5. The mold 64 may be created from a 3D image or negative cast of the patient's extremity. Alternatively, instead of a mold being made from a patient's scan or cast, the mold may correspond to a pre-fabricated size. Thus, a patient may choose a pre-fabricated standard size that most closely fits their foot size. The correct size is determined by measuring the instep of a patient by placing a flexible tape around the instep and heel with the ankle at 90 degrees. The measurement and patient's shoe size are used to determine the best pre-fabricated size on a sizing chart.

In a typical fabrication approach, the mold 64 to be used for the device being fabricated is mounted in a horizontal, tubular vacuum fixture. In a step C, a closed cell polyethylene foam lining material (for inner layer 34) is heated in sheet form and draped over the mold, from the posterior around to the anterior, thus establishing a straight seam from the toe through the instep and up past the proximal edge of the mold, thus enabling a seal around the tubular vacuum fixture. Vacuum is applied, creating an intimate capture by the closed cell polyethylene lining material 34 of the mold 64, as shown in FIG. 5.

An alternate method incorporates a slightly altered mold geometry, designed to accommodate mounting on a vacuum table, where the closed cell polyethylene foam lining material is mounted in a frame, heated, and brought down over the mounted mold. The closed cell polyethylene foam lining material seals against the table which is connected to a similar vacuum source, thus creating a similar seam and intimate capture of the mounted mold by the closed cell polyethylene lining material.

In a step D, after the foam liner (inner layer 34) has cooled sufficiently, strategic areas of the closed cell polyethylene liner are cut away from the mold 64 to expose the anterior foot area 66, the proximal surface 68 of the mold 64, and an anterior strip 70 approximately 1.25-inches (3.2 cm) wide extending from the anterior foot area 66 to approximately ½-inch (1.3 cm) from the proximal edge 44. The anterior portion of the foam liner is cut away by following a trim line that begins on the medial side at a point that bisects the mid-tarsal joint and continues to the lateral side to the bisection of the base of the fifth metatarsal shaft. The material is removed anterior of this trim line such that the resulting brace contains no foot portion forward of the metatarsals. The posterior portion of the boot is cut away by following a trim line from the plantar surface at the calcaneal shelf to a point just proximal the talus. The boot material is removed posterior of this trim line such that the resulting brace contains no heel portion. These cut outs provide for sufficient vacuum for subsequent thermoforming steps as well as reducing bulk in the instep region where the finished device will overlap.

In a step E, the key structural thermoplastic components are manufactured using predetermined, standardized sizing and can be stamped out of sheet material using a clicker die press process or similar. The appropriate size of the horizontal/proximal key structural thermoplastic component (proximal reinforcement strip 46) can be determined by taking a measurement from one anterior/posterior midline to the other anterior/posterior midline, at the location to which the component 46 will be mounted as per the fabrication standards established. The appropriate size of the key structural thermoplastic component, the medial stirrup-type reinforcement strip 40, can be determined by placing a thin, flexible, transparent template identical in dimension to the key thermoplastic component on the mold being used for fabrication. The template that fits the fabrication standards established corresponds to the size of the key structural thermoplastic component 40 used for fabrication.

In a step F, both key structural reinforcement components 40, 46 are heated to approximately 225° F. or until they become moldable. The actual temperature required can vary as a function of the thermoplastic material being used for the key structural thermoplastic components. After heating, the components 40, 46 are removed from the oven and, as illustrated in FIGS. 5 and 6, placed, nearly simultaneously, onto the closed cell polyethylene liner material (inner layer 34), in locations outlined by the fabrication standard. A heat resistant plastic or silicone bag is placed over the entire mold assembly and sealed to the vacuum pipe, at which point vacuum is applied, thus creating an intimate relationship between the bag, the thermoplastic key structural reinforcement components 40, 46 and the mold 64 and closed cell polyethylene liner 34 assembly. Vacuum is applied until the thermoplastic components 40, 46 have cooled to a non-moldable temperature, at which point the vacuum bag is removed from the assembly. During this process, the key structural reinforcement thermoplastic components 40, 46 will have bonded permanently to the closed cell polyethylene lining material 34. The bases of the lace loops 54a are placed on the proximal section 62b (which would typically reside outside the shoe) of the medial stirrup-type reinforcement strip 40. Alternatively, Velcro fasteners can be placed on the exterior of the brace body 12 on the proximal section 62b. In an embodiment, the bases of the lace loops 54a are heat welded or ultrasonic welded together.

In a next step G, the polyolefin elastomeric material to form the outer layer 36 is thermoformed over the assembly, typically using a method nearly identical to the process used for the closed cell polyethylene lining material 34. During thermoforming, the polyolefin elastomeric material outer layer 36 is heated to a temperature from 225° F. to 250° F. and placed over the mold 64, the key structural thermoplastic components 40, 46, and the lace loops 54a to produce the brace body 12. In an embodiment, after the polyolefin elastomeric material for the outer layer 36 is draped over the mold 64 from the posterior around to the anterior thus establishing a straight seam from the region of the toe 66, through the instep (sole portion 22) and up past the proximal edge 68 of the mold 64, a vacuum is used to seal the polyolefin elastomeric material outer layer 36 over the mold, the key structural thermoplastic components 40, 46, and the lace loops 54a. The vacuum can be maintained until the polyolefin elastomeric material outer layer 36 returns to room temperature. The mold 64 is then removed once the polyolefin elastomeric material outer layer 36 has cooled to room temperature to produce the ankle support brace 10.

In a next step H, finishing and trimming of the plastic layers of the brace body is performed.

In a next step I, a cut is made in the polyolefin elastomeric material outer layer 36 across the width of the distal base of a lace loop 54a just dorsal to the stirrup-type reinforcement strip 40, from which the lace loop 54a is pulled through to partially expose the lace loop 54a for future lacing. Fasteners 72 are added to further secure the base of the lace loops 54a, outer layer 36, the stirrup-type reinforcement strip 40, and the foam inner layer 34 together. Alternatively, metallic or non-metallic “boot hook” style hardware can be installed in the proximal section 62b of the medial stirrup-type reinforcement strip 40 (which would typically reside outside the shoe) in place of lace loops.

In an embodiment, the height of the anterior and dorsal surfaces of the mold are such that the medial front edge 18 and the lateral front edge 20 may be overlapped when the ankle support brace is tightened by the closing mechanism 32, as illustrated in FIGS. 1 and 2.

Additionally, in an embodiment, the closed cell polyethylene lining material (inner layer 34), the key structural reinforcement components 40, 46, and the base of the lace loops 54a are laminated in the brace body 12 such that a layer of a synthetic fabric (e.g., Clarino or Lycra) is first placed on the mold, or use a pre-laminated foam with a bonded synthetic layer, followed by the placement of the polyethylene foam inner lining material (34), the key structural reinforcement components 40, 46, and the base of the lace loops 54a. Then the outer layer 36 of polyolefin elastomeric material is placed over the reinforcement strips 40, 46 and lace loops 54a thereby encapsulating the key structural reinforcement components and lace loops in the brace body. Other closure mechanisms besides lace loops and laces are contemplated and considered within the scope of the disclosure.

It is specifically intended that the present disclosure not be limited to the embodiments and illustrations contained herein, but include modified forms of those embodiments including portions of the embodiments and combinations of elements of different embodiments as come within the scope of the following claim.

We claim:

1. An ankle support brace, comprising:
a stirrup-shaped brace body comprising a polyolefin elastomeric material, the brace body comprising:
a back portion;
a medial side portion having a medial front edge;
a lateral side portion having a lateral front edge;
a sole portion having a heel opening edge;
a proximal edge;
a first reinforcement strip with a first end situated on the medial side portion below the proximal edge and extending as a continuous strip along the medial side portion, under the sole portion and along the lateral side portion, with a second end situated on the lateral side portion below the proximal edge;
the medial front edge and the lateral front edge being configured for an overlapping arrangement; and
a closure mechanism to tighten the ankle foot orthosis on a foot.

2. The ankle support brace of claim 1 having no heel portion.

3. The ankle support brace of claim 2 having no foot portion forward of the metatarsals.

4. The ankle support brace of claim 2, further comprising a second reinforcement strip positioned below the proximal edge with a first end situated on the medial side portion and extending along the back portion with a second end situated on the lateral side portion.

5. The ankle support brace of claim 4, wherein the second reinforcement strip is positioned in at least a partially parallel orientation to the proximal edge.

6. The ankle support brace of claim 4, wherein the first and second reinforcement strips comprise a polyolefin elastomeric material.

7. The ankle support brace of claim 2, comprising a closed cell foam inner lining bonded to a polyolefin elastomeric material outer layer, with the first second reinforcement strip situated between the inner lining and the outer layer.

8. The ankle support brace of claim 7, further comprising a material layer bonded to the foam inner lining.

9. The ankle support brace of claim 8, wherein the material layer is selected from the group consisting of a synthetic leather material and a synthetic elastic fabric.

10. The ankle support brace of claim 2, wherein the closure mechanism comprises laces, hooks, loops, Velcro, elastic, fasteners, or a combination thereof.

11. The ankle support brace of claim 2, wherein the closure mechanism comprises laces and lace loops.

12. The ankle support brace of claim 11, wherein the lace loops have a base in contact with the first reinforcement strip.

13. The ankle support brace of claim 11, wherein the closure mechanism further comprises boot hooks mounted on the first reinforcement strip, the second reinforcement strip, or both.

14. The ankle support brace of claim 3, being structured for insertion into a shoe.

15. The ankle support brace of claim 3, where the sole portion is in a plane at least partially horizontal to the medial and lateral side portions.

16. The ankle support brace of claim 3, wherein the sole portion is at least partially perpendicular to at least a portion of the medial side portion, at least a portion of the lateral side portion, or both.

17. An ankle support brace, comprising:
a stirrup-shaped brace body comprising a foam inner lining bonded to a thermoplastic material outer layer, and at least one reinforcement strip situated between the inner lining and the outer layer;
the at least one reinforcement strip as a continuous element extending from a medial side portion, under a sole portion comprising a heel opening edge and to a lateral side portion, the sole portion.

18. The ankle support brace of claim 17 having no heel portion and no foot portion forward of the metatarsals.

19. The ankle support brace of claim 18, wherein the foam material comprises a closed cell foam material.

20. The ankle support brace of claim 18, wherein the sole portion is in a plane at least partially horizontal to the medial side portion and the lateral side portion.

21. The ankle support brace of claim 20, wherein the sole portion is at least partially perpendicular to at least a portion of the medial side portion and at least a portion of the lateral side portion.

22. The ankle support brace of claim 21, further comprising a second reinforcement strip positioned below a proximal edge of the brace body, with a first end situated on the medial side portion and extending along the back portion, and with a second end situated on the lateral side portion.

23. A method of manufacturing the ankle support brace of claim 1, the method comprising:
forming a foam material over a mold of a lower extremity comprising a heel portion and a foot portion forward of the metatarsals;
removing the foam material from the heel portion and foot portion forward of the metatarsals to form the foam material into a stirrup-like shape;
attaching the first thermoplastic reinforcement strip component onto the foam material;
forming an outer layer of a polyolefin elastomeric material over the foam material and the reinforcement strip component;
setting the closure mechanism into the outer layer, and
removing the mold to produce the ankle foot orthosis.

24. The method of claim 23, further comprising attaching a second thermoplastic reinforcement strip component onto the foam material below the proximal edge with a first end situated on the medial side portion and extending along the back portion with a second end situated on the lateral side portion.

25. The method of claim 23, further comprising placing a base of a lace loop on the first reinforcement strip before forming the outer layer of the polyolefin elastomeric material.

26. The method of claim 25, further comprising adding a fastener to secure the base of the lace loop to the first reinforcement strip and the foam material.

27. The method of claim 25, further comprising cutting the outer layer of the polyolefin elastomeric material to expose the lace loop.

28. The method of claim 25, further comprising placing a shoelace through the lace loop.
